(19) United States
(12) Patent Application Publication
(10) Pub. No.: US 2026/0277830 A1
(43) Pub. Date: Sep. 17, 2026

(54) MEMORY-EFFICIENT DATA FORMAT AND UTILIZATION FOR PARALLEL PROCESSING

(71) Applicant: Roche Sequencing Solutions, Inc., Pleasanton, CA (US)

(72) Inventor: Charles Seberino, Gilbert, AZ (US)

(21) Appl. No.: 19/669,098

(22) Filed: May 6, 2026

Related U.S. Application Data
(63) Continuation of application No. PCT/US2024/054535, filed on Nov. 5, 2024.
(60) Provisional application No. 63/596,370, filed on Nov. 6, 2023.

(51) Int. Cl. G06F 13/16

(52) U.S. Cl. CPC G06F 13/1647; G06F 13/1615

(57) ABSTRACT

A method for efficiently processing data using a parallel processor includes storing in memory data in an array in an interleaved format. The first two bits of data stored in the memory for each read is accessed using a plurality of threads, with each thread processing data from one sequence read. Each thread in parallel performs the same operation on the accessed first two bits of data. These steps are repeated until all the data in the array is processed.

### CROSS-REFERENCE TO RELATED APPLICATIONS

This application is a continuation of International Patent Application No. PCT/US2024/054535, filed Nov. 5, 2024, which claims priority to U.S. Provisional Application No. 63/596,370, filed on Nov. 6, 2023, each of which is hereby incorporated by reference in its entirety for all purposes.

### INCORPORATION BY REFERENCE

All publications and patent applications mentioned in this specification are herein incorporated by reference to the same extent as if each individual publication or patent application was specifically and individually indicated to be incorporated by reference.

### FIELD

This application relates generally to systems and methods to efficiently access and utilize data in memory, and more specifically to methods to efficiently access and utilize data with parallel processing hardware, such as a GPU and/or multiple CPUs.

### BACKGROUND OF THE INVENTION

One of the challenges with parallel processing is the ability to efficiently access and utilize data in memory for calculations. This can take a variety of forms including a) access time (to load or store), b) data access patterns (contiguous, strided, random), c) cache line size, and d) minimum required memory to start or continue processing, to name a few. Parallel processing hardware, such as a GPU, will have fixed behavior for some of these, and it is up to the developer to abide by and use them as efficiently as possible. Another challenge for parallel programming, and software development generally, is to create algorithms that are easy to describe and understand. The invention detailed here provides a simplification to code development. Once initialized, it provides a guaranteed mechanism of data access on a per-thread basis. Writing clean code, that is easy to understand, bug free, and high performance is difficult. While manual methods can maintain state and provide correct indexing of data arrays, they necessarily add complexity to the algorithm implementation and introduce potential for mistakes. By taking advantage of an encapsulated, dedicated mechanism that is purpose-built for manipulating arrays in parallel, the developer is able to focus more on the algorithm rather than the mechanics of memory access.

### SUMMARY OF THE INVENTION

The present invention relates generally to systems and methods to efficiently access and utilize data in memory, and more specifically to methods to efficiently access and utilize data with parallel processing hardware, such as a GPU and/or multiple CPUs.

In some embodiments, a method for efficiently processing genomic data using a parallel processor is provided. The method includes storing genomic data in an array in an interleaved format, wherein the genomic data comprises a plurality of sequence reads; loading a first eight bits of data from each read into a local memory; accessing in parallel a first two bits of data stored in the local memory for each read using a plurality of threads, with each thread processing data from one sequence read; performing an operation on the accessed first two bits of data with each thread in parallel, wherein the plurality of threads are performing the same operation; repeating the accessing and performing an operation steps on the remaining data stored in the local memory until the entire eight bits has been processed by each thread; loading successive eight bits of data from each read into the local memory; processing the successive eight bits of data in a same manner as the first eight bits of data; and repeating the loading and processing steps until all the data in the array is processed.

In some embodiments, each thread loads the successive eight bits into memory using a stride of the array to access the successive eight bits.

In some embodiments, a method for efficiently processing genomic data using a parallel processor is provided. The method includes storing genomic data in an array in an interleaved format, wherein the genomic data comprises a plurality of sequence reads; accessing in parallel a first two bits of data stored in the local memory for each read using a plurality of threads, with each thread processing data from one sequence read; performing an operation on the accessed first two bits of data with each thread in parallel, wherein the plurality of threads are performing the same operation; and repeating the accessing and performing an operation steps on successive two bits of data until all the data in the array is processed.

In some embodiments, each thread loads the successive two bits into memory using a stride of the array to access the successive two bits.

In some embodiments, a method for efficiently processing data using a parallel processor is provided. The method includes storing data in an array in an interleaved format, wherein the genomic data comprises a plurality of sequence reads; accessing in parallel a first two bits of data stored in the local memory for each read using a plurality of threads, with each thread processing data from one sequence read; performing an operation on the accessed first two bits of data with each thread in parallel, wherein the plurality of threads are performing the same operation; and repeating the accessing and performing an operation steps on successive two bits of data until all the data in the array is processed.

In some embodiments, each thread loads the successive two bits into memory using a stride of the array to access the successive two bits.

In some embodiments, a computer product comprising a computer readable medium storing a plurality of instructions for controlling a computer system to perform an operation of any of the methods herein.

In some embodiments, a system includes the computer product described herein; and one or more processors for executing instructions stored on the computer readable medium.

In some embodiments, a system includes means for performing any of the methods herein.

In some embodiments, a system is configured to perform any of the methods described herein.

In some embodiments, a system includes modules that respectively perform the steps of any of the methods described herein.

### BRIEF DESCRIPTION OF DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A illustrates one example of an array for storing data.

FIG. 1B illustrates one example of an array for storing genomic data in an interleaved format.

FIG. 2 illustrates another example of an array for storing genomic data in an interleaved format.

FIG. 3 illustrates an example of a sizing array derived from the array illustrated in FIG. 2

FIG. 4 illustrates a packed array derived from the array illustrated in FIG. 2.

FIG. 5 illustrates an offset array derived from the array illustrated in FIG. 2.

FIG. 6 illustrates an example of an array for storing genomic data for use with TwoBitPointer.

FIG. 7 illustrates a sizing array derived from the array illustrated in FIG. 6.

FIG. 8 illustrates a read length array derived from the array illustrated in FIG. 6.

FIG. 9 is a computer system, according to certain aspects of the present disclosure.

### DETAILED DESCRIPTION OF THE INVENTION

A Graphical Processing Unit, or GPU, is a massively parallel computing resource supporting Single Instruction Multiple Data (SIMD) execution. Typically composed of several thousand cores, a GPU is able to keep tens or hundreds of thousands of threads of execution in flight. In order to achieve this high level of parallelism, the memory used for computation should also be aligned in a data parallel fashion—where the data is structured so that the memory controller on the GPU can efficiently fetch for many threads simultaneously.

In other embodiments, the computing resource can use one or more central processing units (CPUs). For example, the systems and methods described herein can be used on a CPU system with CPU Vector intrinsics (i.e., SSE instructions, AVX512) where the CPU acts similarly to a vector/parallel processor.

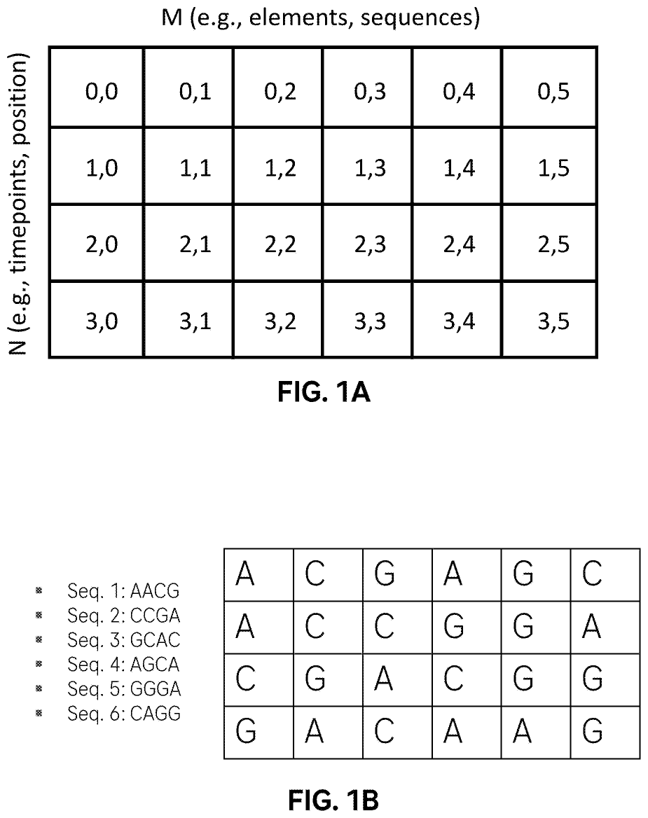

As shown in FIG. 1A, data can be stored in a 2D matrix of size [x, y], where the x dimension, i.e., the data stored in a row, includes the data to be processed simultaneously in parallel by the plurality of threads. The y dimension stores the next data to be processed by the threads. For example, sensor data (e.g., adc counts, voltage, current, resistance, etc.) from 6 sensors with each sensor generating 4 data points can be stored in a matrix with 4 rows and 6 columns (i.e., a [4, 6] matrix), with each column containing data from one sensor and each row containing data from all 6 sensors at one time point. Another example would be storing the base call reads of 6 sequences, with each sequence 4 bases long. Again, the data can be stored in a matrix with 4 rows and 6 columns (i.e., a [4, 6] matrix.

Organizing the data in this manner results in interleaved data. FIG. 1B illustrates how data from 6 sequences can be stored in an interleaved format. For example, the first base from each read is stored in the first row of the matrix. The second base in each read is stored in the second row of the matrix. The third base in each read is stored in the third row in the matrix. The fourth base in each read is stored in the fourth row in the matrix. This results in the data from each sequence stored in columns. For example, Seq. 1 is stored in column 1, Seq. 2 is stored in column 2, and so on.

In the matrix representation above and illustrated in FIGS. 1A and 1B, each column of data can be processed in parallel using a single thread because the data has been interleaved. The interleaved format results in the first data point of each element being placed in the first row, which can be retrieved by a memory fetch and each data point can be sent to a thread to be processed in parallel.

In contrast, if the data was not stored in an interleaved manner, for example if the sequences were stored in rows instead of columns, a memory fetch would retrieve all the data from the first sequence but none of the data from the other sequences. Because the goal is for each thread to process the data of one sequence, the memory fetch in the non-interleaved data only retrieves data for one thread, which means multiple memory fetches must be used in order to pass data to each thread before all the threads are processing data. In contrast, when the data is interleaved, all the threads are processing data after the first memory fetch. Subsequent memory fetches then can continuously feed all the threads new data to process, thereby reducing the bottlenecks that can occur from a mismatch between the memory fetch operations and the thread operation steps.

In the matrix representation above, 6 threads are each simultaneously performing the same computation using the data along a column: f(x, y)=f(0,0)⊗f(1,0)⊗f(2,0)⊗f(3,0), where ⊗ represents a tensor product. For a matrix with X elements, X threads can be used to simultaneously perform the same computation using the data along a column, with one thread assigned to one column, such that the entire matrix of data is processed simultaneously. If there are more elements than threads, a subset of data in the matrix can be processed simultaneously using all threads until completed and then the next subset can be processed. For example, if there are 32 elements and 16 threads, the data can be divided into two subsets of 16 elements, which can be processed sequentially.

In other embodiments, the data can be stored in higher dimension matrices, such as a 3D matrix or even higher dimension matrices. An example of a 3D matrix is described below.

### WorkerPointer

One of the challenges in writing software to perform the above computation is to ensure that each thread only accesses the data along a single column. When the above matrix, shown in FIG. 1A is represented as a 1D array, accessing element [1,1] is calculated in 1D as [6*1+1] or index 7. More generally, the index x for accessing element [i, j] in a matrix of size [X, Y] is calculated as [i*Y+j], wherein i goes from 0 to (X−1) and j goes from 0 to (Y−1). In the absence of the invention described herein, the developer is responsible for updating and recomputing the index. However, by utilizing the interleaved data format along with the WorkerPointer and TwoBitPointer format described below, the index is automatically handled.

In some embodiments, a class called WorkerPointer contains information for working in a parallel fashion with data arrays that are formatted as described herein. WorkerPointer also provides easy iteration over the data in a safe and methodical manner. Using WorkerPointer only requires passing it the underlying data array pointer and the stride or Y dimension of the array.

```
  // Previously allocated data with dimensions ‘numRows‘ and ‘numColumns‘ and 8-
bit type.
  // uint8_t data[numRows][numColumns];
  // Construct a WorkerPointer to facilitate iterating along columns.
  WorkerPointer<TwoBit> worker(data, numColumns);
  // In this example a simple column sum is performed. The computation described in
this example is just one type of operation that can be performed and not the focus of this
example.
  // The main point illustrated by this example is that the iterator simplifies access to a
single column which
  // has the benefit of keeping the developer from having to keep track of the
  // proper index and offset into the data array. The algorithm implementation is
  // also simplified by not having to worry about striding.
  // The loop below can be done in parallel efficiently where each thread would
  // selects a different column to process.
  int columnSum = 0;
  int columnToProcess = 1;
  // Creation of the iterator is cheap.
  auto iter = worker.begin(columnToProcess);
  // Perform automatic end-of-column check.
  while (iter != worker.end( )) {
  // Do something with *iter, which is the value in column 1 at the current row.
  columnSum += *iter;
  // Increment the iterator to the next row, automatically handling striding.
  ++iter;
  }
  int totalSize = numElements * numTimePoints;
  // Allocate ‘data‘ array of size ‘totalSize‘ and populate.
  // unsigned char* data = ...
  // ...
  // Constructs a Worker using a 1D data array of size ‘totalSize‘
  // and X dimension of ‘numElements‘.
  WorkerPointer worker(data, totalSize, numElements);
  // We can now sum over all the data in column 1 with:
  int sum = 0;
  auto iter = worker.begin(1);
  for (; iter != worker.end( ); ++iter) {
    sum += *iter;}
```

The implementation above can be simultaneously run across many threads, where each thread operates on a particular element in the array. End of array checking is performed automatically and the data is naturally accessed in a memory efficient manner across all active threads, as further explained below.

When the WorkerPointer (or TwoBitPointer, described below) first starts, it will perform a memory fetch to initialize the local cache. On the GPU all active threads (in groups of 32, for example) will issue that command simultaneously and the hardware memory controller will determine that it can satisfy all requests with a single memory fetch (say of 128 bytes). For the 2-bit case, the cache value will be used for the next 3 accesses and on the fourth request, will make another memory fetch to load the next set of elements.

Interleaving data is an efficiency gain for parallel programming. Caching of the data is a feature of the TwoBitPointer to reduce the frequency of expensive memory fetches. The current 8-bit cache saves 3 out of 4 fetches. Increasing it to a 16-bit cache would reduce 7 out of 8 fetches, at a potential cost of having to store unused values at the end of the valid data array when not a multiple of 8.

Although this example shows summation as the operation performed by the worker, any other operation can be performed by the worker, such as accessing to check against a value for a searching function.

Other operations that can be performed by threads efficiently using the data format and methods described herein (e.g. WorkerPoint and TwoBitPointer), include:

Taking the output of a neural network or other algorithm or software code and using the TwoBitPointer (described further below) or WorkerPointer to store the result in an efficient manner. TwoBitPointer can be used especially efficiently for output or data that is two bit in size, although it can be extended for use with data of other sizes, as further described herein.

Iterating over raw ADC values using WorkerPointer to perform various operations, such as summation and/or storing the values.

Some classes of operations performed are:

Statistical calculations during basecalling—sum/mean/min/max/rate/quality—both per sequencing cell of an array of cells of a sensor chip and then aggregated over the entire sensor chip.

Matching or searching for a particular pattern. In sequencing applications, 4 bases can be stored into a byte (i.e., 8 bits) of memory, which then allows the examination and/or matching of 4 bases at a time rather than just one at a time.

Consensus—assembling multiple basecall reads together and examining them in aggregate to determine the “one true” sequence (i.e., the consensus sequence), thereby removing sequencing-induced errors. Each column is a base position in the genome being examined and/or corrected.

General scatter-gather: Can take a packed representation and expand it out (scatter) or perform the reverse (gather). Summation is one type of gather/reduction computation.

These operations can also be used with TwoBitPointer and any of the methods described herein.

### TwoBitPointer

TwoBitPointer is a specialized class of WorkerPointer that is designed to work on values that are 2-bits in size. This is particularly useful since computers naturally store data in groups of 8-bits. In an 8-bit system, there would be four individual accesses of the same byte of memory needed to pull the 4 consecutive 2-bit values. As in many hardware architectures, memory fetching into local or register locations can take significantly longer than any operations on the values. So efficient memory access is very important. In general, loading bits into “local cache” refers to loading the bits from global memory into register memory. This makes it instantly accessible as it is the fastest access location available on any hardware device. A general view from fastest to slowest memory is: Register, L1 cache, L2 cache, . . . L<N> cache, and finally global memory. An order of magnitude speed increase is typical between the different levels.

This WorkerPointer specialization locally caches 8-bit values per element to keep access time down. This allows 4 2-bit retrievals to be performed using the faster local cache rather than performing all the retrievals from a slower memory storage device, such as a solid state drive (SSD). This is true for both reading and writing operations. Use of this class is no different than the standard case, as shown in the example below:

// The source array is still allocated with an unsigned byte
// as the underlying datatype.
int totalSize = numElements * numTimePoints;
// Allocate ‘data‘ array of size ‘totalSize‘ and populate.
// unsigned char* data = malloc(totalSize);
// ...
// Construct a TwoBit WorkerPointer similar to before.
WorkerPointer<TwoBit> worker(data, totalSize, numElements);
// Iteration and usage is the same.
int sum = 0;
auto iter = worker.begin(1);
for (; iter != worker.end( ); ++iter) {
sum += *iter;
}

Very little changes when comparing the TwoBitPointer version to the standard WorkerPointer version. In fact the only significant code change involves providing a template parameter of TwoBit to the WorkerPointer constructor. The TwoBit class provides the underlying efficient access of a single 8-bit value into its 4 constituent 2-bit values. As described above, this 8-bit value is cached in local memory for rapid access. This speeds up the process because memory fetching on modern computers generally requires the most time, compared to compute, and systems employ multiple levels of memory caching (register, L1, L2, L3, etc) to prefetch or hold onto often-used memory spaces to speed up work. Without caching, a naive approach would be to read each of the 2-bits separately each time, incurring 4× the number of global memory reads.

In addition, for data stored in other sizes not divisible by 8 or that do not fit neatly into 8 bits, such as data that is encoded in 3 bits, 24 bits (3×8) can be retrieved and stored locally in register memory so that there is no wasted space from partially retrieved data. More generally, the amount of bits to be retrieved and stored locally is the least common multiple of the unit size of the data (i.e., 2 bit data, 8 bit data, 16 bit data, etc.) and 8.

In other embodiments, a larger amount of bits can be stored on the local cache, even for 2 bit data, such as using 16 bit, 32 bit, 64 bit, etc.

### Example 1

The following is an example of C++ source code performing a simple summation of timepoint data using a single thread:

```
// Results array has one value per element.
int results[numElements];
// Data array is 2D with time along X and elements along Y.
int data[numElements][numTimePoints];
// Assume the data array has been initialized with valid contents to sum.
// Loop through each element, summing timepoints.
for (int row = 0; row < numElements; ++row) {
  results[row] = 0;
  for (int col = 0; col < numTimePoints; ++col) {
    results[row] += data[row][col];
  }
}
```

This code performs a reduction of the numTimePoints (X) dimension and places the sum into the results array, with one value per element.

### Example 2

A variation of the above, but using a 1D pointer instead of 2D looks like this:

```
  // Results array has one value per element.
  int results[numElements];
  // Data array is logically 2D, but composed from a 1D array. Time along X and
elements along Y.
  int data[numElements * numTimePoints];
// Assume the data array has been initialized with valid contents to sum.
  // Loop through each element, summing timepoints.
  for (int row = 0; row < numElements; ++row) {
    results[row] = 0;
    for (int col = 0; col < numTimePoints; ++col) {
      results[row] += data[row * numTimePoints + col];
    }
}
```

In the above code, the user is responsible for ensuring that the 2D-to-1D indexing is being performed correctly.

### Example 3

The next example is a variant of the above, but utilizes a vector container class from the Standard Template Library (STL) for ease of use.

```
  // Results array has one value per element. Init to 0.
  std::vector<int> results(numElements, 0);
  // Data array is logically 2D, but composed from a 1D array. Time along X and
elements along Y.
  std::vector<int> data(numElements * numTimePoints);
  // Assume the data array has been initialized with valid contents to sum.
  // Loop through each element, summing timepoints.
  int index1D = 0;
  int row = 0;
  for (auto it = data.cbegin( ); it != data.cend( ); ++it) {
    results[row] += *it;
    // Update row value based on the integer multiple of
    // numTimePoints.
    ++index1D;
    if (0 == (index1D % numTimePoints)) ++row;
  }
```

The above code simplifies looping over the data array by using an iterator, which also provides out-of-bounds checking.

In all of the examples and embodiments described herein, the data can be laid out so that a memory controller would be able to efficiently load (and potentially cache in anticipation) subsequent memory requests. It is considered “cache friendly”. This is true for serialized code where only a single element is being operated on at a time before proceeding to the next element. For example, the timepoints or other array elements can be contiguous in memory.

### Example 4

A parallelized implementation is shown below using a C++17 parallel execution policy feature.

```
  // Results array has one value per element. Init to 0.
  std::vector<int> results(numElements, 0);
  // Data array is logically 2D, but composed from a 1D array. Time along X and
elements along Y.
  std::vector<int> data(numElements * numTimePoints);
  // Assume the data array has been initialized with valid contents to sum.
  // Create a lambda expression(function) to perform the looping
  // over a single element's collection of timepoints.
  auto lambda = [&] (int& res) {
    int sum = 0;
    // Compute row using pointer difference of the incoming
    // reference element with the starting result array pointer.
    int row = &res − results.data( );
    // Create starting and ending locations for this element.
    auto it = data.begin( ) + row * numTimePoints;
    auto itEnd = it + numTimePoints;
    for (; it != itEnd; ++it) {
      sum += *it;
    }
    return sum;
  };
  // Compute each element in parallel with no strict ordering.
  std::for_each(std::execution::par_unseq, results.begin( ),
        results.end( ), lambda);
```

In this example, when performing work using a parallel approach, multiple elements can be operated on simultaneously, and for that the per-element values would be contiguous in memory.

### Example 5

The following is an example of the data formats that can be used in sequencing applications, and particularly for handling variable length output from a sequencer.

### Terminology

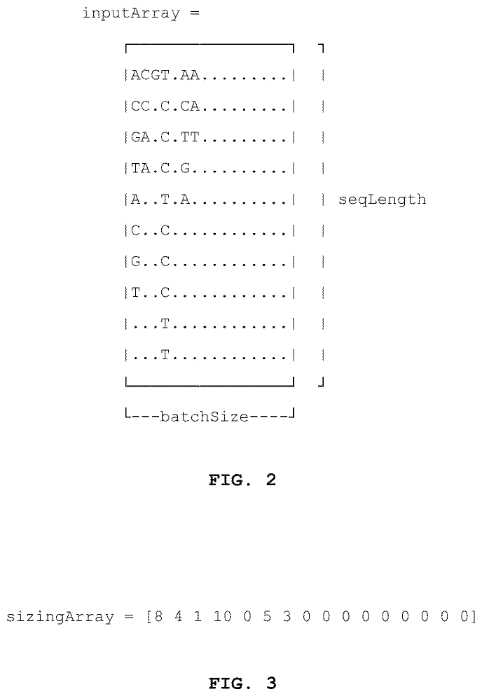

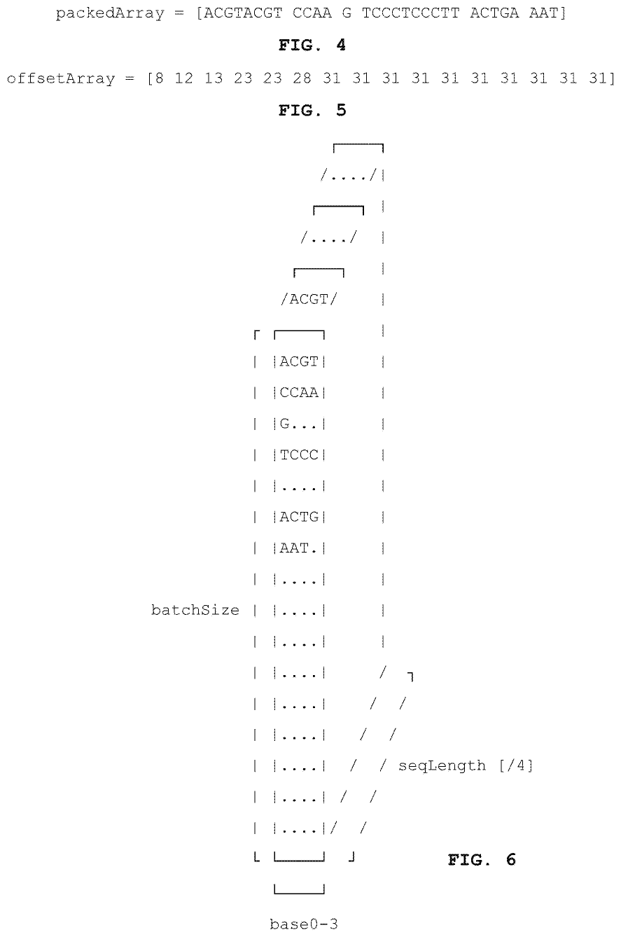

These terms are used throughout the document and are defined here for clarity. The first two correspond to neural network terms used to describe input data that is reused in a generalized context.
‘batchSize’—Denotes the number of unique input samples to be processed concurrently. For DNA data, it would typically represent the number of sequencing reads. Typically a power of 2, with a nominal value of 64 k. Powers of 2, or more loosely, multiples of 8, are sizes that are highly optimized on most hardware. Cache sizes, hardware registers, etc. are almost always a power of 2, so then having the data also be in this granularity lets the hardware and software be well aligned, thereby improving performance.
‘seqLength’—The length (typically max length) of a complete read. In some embodiments, seqLength is set at about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 bases.
‘inputArray’—An expanded (full), column-major, interleaved array. In some embodiments, the array can be 1D, 2D, or 3D. In the context of DNA data, the array can be in the shape [batchSize x seqLength], where ‘batchSize’ is the fastest changing dimension. This naturally supports efficient memory coalescing in GPU kernels across threads. FIG. 2 gives an example containing 6 samples in an array of size [16×10] where the ‘.’ are unspecified values.
Since this is a full array, a ‘sizingArray’ is used jointly to keep track of the number of valid elements.
‘sizingArray’—Accompanying array that provides a per-sample count of the number of valid elements available. For example, the sizingArray can be a uint32 array of size ‘batchSize’—one value per sample, as shown in FIG. 3.
‘packedArray’—A compacted representation of ‘inputArray’, but in transposed form. Elements and samples that are unspecified are removed. Typically used when writing to disk. An example of the packedArray’ is shown in FIG. 4. Note that spaces have been added to packedArray for ease of interpretation, but are not present in the actual data.
‘packedArray’ is a contiguous 1D array using an accompanying ‘offsetArray’ to parallelize per-sample reading and writing.
‘offsetArray’—Similar to ‘sizingArray’, but represented as a cumulative sum, where each element is the ending offset into the data array for each sample. Taking the difference ‘offsetArray[N]−offsetArray[N−1]’ converts the values back into the ‘sizingArray’ representation. An implied ‘0’ value is assumed when back-converting the first element. The last value in the array can always be used to retrieve the total amount of data in ‘packedArray’. An example of the ‘offsetArray’ is shown in FIG. 5.
‘frameSize’—corresponds to the total number of input samples to be processed. When processing, this input data is broken up into ‘batchSize’ chunks to be fed into to downstream analysis. Padding may be applied on the last chunk to guarantee a fixed memory size.

### Implementation Classes

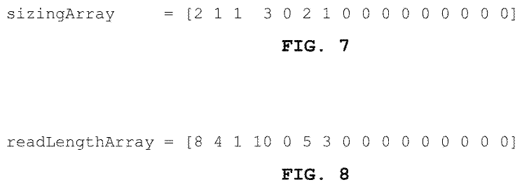

**Pointer**—Provides ‘std::vector’—like behavior in host and device code. Specifically, it is a pointer to a contiguous array of data and an associated value for the number of elements contained within. It doesn't manage the memory directory. Allocations, whether host or device-based, are required. Constructors, iterators, accessors, and mutator methods are provided for convenience. It contains 2 member variables:
‘storage_’—templated type raw pointer
‘storagSize_’—number of element available in ‘storage_’ (size_t)
WorkerPointer**-As described above and further described here, WorkerPointer is a convenience class that provides per-sample iterator access to an underlying ‘Pointer’. Typical use is to treat a ‘Pointer’ as a 2D array with fixed dimensions, although it does have rudimentary support for 3D striding. Provides forward and reverse iterator support as well as random access and const access. Basic use is to pass a sample index to a ‘begin( )’, ‘cbegin( )’, or ‘rbegin( )’ method, which all return a usable iterator to work with. This ‘WorkerPointer’ provides a valuable mechanism used in many device kernels that work on a single thread per sample. It contains 2 member variables:
‘ptr_’ Holds a ‘Pointer’ to the underlying data
‘frameSize_’ Per-sample striding value
**TwoBitPointer**—As described above and further described here, TwoBitPointer is a variant of the ‘WorkerPointer’, which provides packing of data taking only 2-bits of space. For example, in sequencing a typical use is for basecall data. Provides methods for forward and reverse iterators as well as internal caching of state for efficient global memory loads and stores. Notionally, a ‘TwoBitPointer’ is represented as a 3D array, as shown in FIG. 6, where the first (and fastest changing) dimension is of length 4. Note that FIG. 6 illustrates the same data as FIG. 2, but in TwoBitPointer format. The data is still interleaved, but each read is stored in 8-bit chunks (4 bases) rather than in 2 bit chunks as shown in FIG. 2. In the context of DNA bases, the encoding is in alphabetical order, where ‘ACGT’ corresponds to the values [0, 1, 2, 3] respectively. Values are stored left-to-right (most significant bye “MSB” ordering) within each byte, although the ordering can alternatively be done using least significant byte (“LSB”) ordering. A separate array as shown in FIG. 7 that is separate from ‘inputArray’ may be needed since this format could have partially packed representation (not a multiple of 4). Where the ‘inputArray’ will give the total number of elements per sample (byte in this case), it is insufficient to describe the number of bases present. For that a separate ‘readLengthArray’ is used to give the per-sample number of bases. The TwoBitPointer version of the sizingArray is illustrated in FIG. 7, and the TwoBitPointer version of the readLengthArray is shown in FIG. 8.

Note that the ‘seqLength’ dimension can be reduced by a factor of 4 to conserve memory.

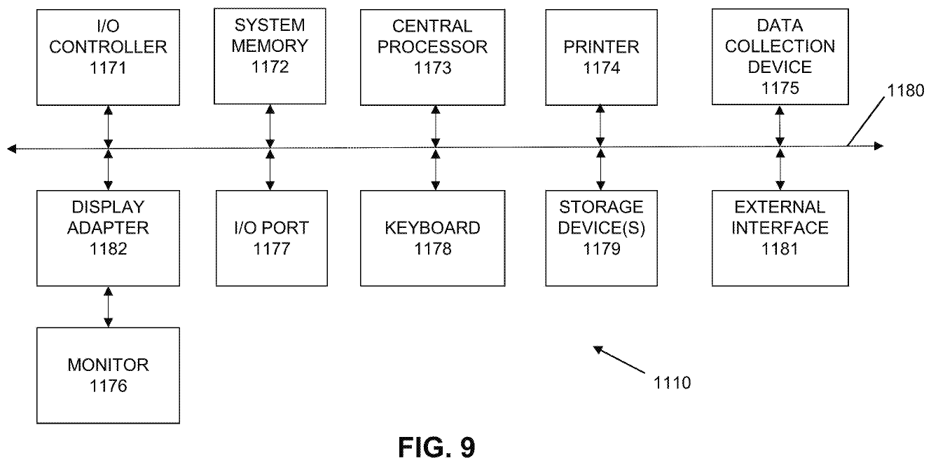

Any of the computer systems mentioned herein can utilize any suitable number of subsystems. Examples of such subsystems are shown in FIG. 9 in computer system 1110. In some embodiments, a computer system includes a single computer apparatus, where the subsystems can be the components of the computer apparatus. In other embodiments, a computer system includes multiple computer apparatuses, each being a subsystem, with internal components. A computer system can include desktop and laptop computers, tablets, mobile phones, and other mobile devices.

The subsystems shown in FIG. 9 are interconnected via a system bus 1180. Additional subsystems such as a printer 1174, keyboard 1178, storage device(s) 1179, monitor 1176 which is coupled to display adapter 1182, and others are shown. Peripherals and input/output (I/O) devices, which couple to I/O controller 1171, can be connected to the computer system by any number of means known in the art such as I/O port 1177 (e.g., USB, FireWire®). For example, I/O port 1177 or external interface 1181 (e.g., Ethernet, Wi-Fi, etc.) can be used to connect computer system 1110 to a wide area network such as the Internet, a mouse input device, or a scanner. The interconnection via system bus 1180 allows the central processor 1173 to communicate with each subsystem and to control the execution of a plurality of instructions from system memory 1172 or the storage device(s) 1179 (e.g., a fixed disk, such as a hard drive, or optical disk), as well as the exchange of information between subsystems. The system memory 1172 and/or the storage device(s) 1179 can embody a computer readable medium. Another subsystem is a data collection device 1175, such as a camera, microphone, accelerometer, and the like. Any of the data mentioned herein can be output from one component to another component and can be output to the user.

A computer system can include a plurality of the same components or subsystems, e.g., connected together by external interface 1181, by an internal interface, or via removable storage devices that can be connected and removed from one component to another component. In some embodiments, computer systems, subsystem, or apparatuses communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g., an APSIC or FPGA) and/or using computer software with a generally programmable processor in a modular or integrated manner. As used herein, a processor can include a single-core processor, multi-core processor on a same integrated chip, or multiple processing units on a single circuit board or networked, as well as dedicated hardware, including a GPU. Based on the disclosure and teachings provided herein, a person of ordinary skill in the art will know and appreciate other ways and/or methods to implement embodiments of the present invention using hardware and a combination of hardware and software.

Any of the software components or functions described in this application can be implemented as software code to be executed by a processor using any suitable computer language such as, for example, Java, C, C++, C#, Objective-C, Swift, or scripting language such as Perl or Python using, for example, conventional or object-oriented techniques. The software code can be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium can be any combination of such storage or transmission devices.

Such programs can also be encoded and transmitted using carrier signals adapted for transmission via wired, optical, and/or wireless networks conforming to a variety of protocols, including the Internet. As such, a computer readable medium can be created using a data signal encoded with such programs. Computer readable media encoded with the program code can be packaged with a compatible device or provided separately from other devices (e.g., via Internet download). Any such computer readable medium can reside on or within a single computer product (e.g., a hard drive, a CD, or an entire computer system), and can be present on or within different computer products within a system or network. A computer system can include a monitor, printer, or other suitable display for providing any of the results mentioned herein to a user.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for efficiently processing genomic data using a parallel processor, the method comprising:
storing genomic data in an array in an interleaved format, wherein the genomic data comprises a plurality of sequence reads;
loading a first eight bits of data from each read into a local memory;
accessing in parallel a first two bits of data stored in the local memory for each read using a plurality of threads, with each thread processing data from one sequence read;
performing an operation on the accessed first two bits of data with each thread in parallel, wherein the plurality of threads are performing the same operation;
repeating the accessing and performing an operation steps on the remaining data stored in the local memory until the entire eight bits has been processed by each thread;
loading successive eight bits of data from each read into the local memory;
processing the successive eight bits of data in a same manner as the first eight bits of data; and
repeating the loading and processing steps until all the data in the array is processed.

2. The method of claim 1, wherein each thread loads the successive eight bits into memory using a stride of the array to access the successive eight bits.

3. A method for efficiently processing genomic data using a parallel processor, the method comprising:
storing in memory genomic data in an array in an interleaved format, wherein the genomic data comprises a plurality of sequence reads;
accessing in parallel a first two bits of data stored in memory for each read using a plurality of threads, with each thread processing data from one sequence read;
performing an operation on the accessed first two bits of data with each thread in parallel, wherein the plurality of threads are performing the same operation; and
repeating the accessing and performing an operation steps on successive two bits of data until all the data in the array is processed.

4. The method of claim 3, wherein each thread loads the successive two bits into memory using a stride of the array to access the successive two bits.

5. A method for efficiently processing data using a parallel processor, the method comprising;
storing in memory data in an array in an interleaved format;
accessing in parallel a first two bits of data stored in the memory for each read using a plurality of threads, with each thread processing data from one sequence read;
performing an operation on the accessed first two bits of data with each thread in parallel, wherein the plurality of threads are performing the same operation; and
repeating the accessing and performing an operation steps on successive two bits of data until all the data in the array is processed.

6. The method of claim 5, wherein each thread loads the successive two bits into memory using a stride of the array to access the successive two bits.

7. A method for efficiently processing data using a parallel processor, the method comprising:
storing data in an array in an interleaved format, wherein the data is encoded in X bits;
loading a first Y bits of data into a local memory, wherein Y is the least common multiple of X and 8;
accessing in parallel a first X bits of data stored in the local memory using a plurality of threads;
performing an operation on the accessed first X bits of data with each thread in parallel, wherein the plurality of threads are performing the same operation;
repeating the accessing and performing an operation steps on the remaining data stored in the local memory until the entire Y bits has been processed by each thread;
loading successive Y bits of data into the local memory;
processing the successive Y bits of data in a same manner as the first Y bits of data; and
repeating the loading and processing steps until all the data in the array is processed.

8. The method of claim 7, wherein the data can be sensor data from a plurality of sensors.

9. The method of claim 7, wherein the data can be sequencing data.
